(19) United States
(12) Patent Application Publication
(10) Pub. No.: US 2025/0195546 A1
(43) Pub. Date: Jun. 19, 2025

(54) CANNABINOID-CONTAINING COMPOSITIONS

(71) Applicant: BUZZELET DEVELOPMENT AND TECHNOLOGIES LTD., Or Akiva (IL)

(72) Inventors: Aharon EYAL, Jerusalem (IL); Noa RAZ, Gizo (IL)

(21) Appl. No.: 18/847,275

(22) PCT Filed: Mar. 14, 2023
(86) PCT No.: PCT/IB2023/052458

§ 371 Date: Sep. 16, 2024

Related U.S. Application Data
(60) Provisional application No. 63/320,222, filed on Mar. 16, 2022.
(60) Provisional application No. 63/321,782, filed on Mar. 21, 2022.

(51) Int. Cl. A61K 31/00; A24B 15/24; A24B 15/26; A24B 15/30; A61K 36/185

(52) U.S. Cl. CPC A61K 31/658; A24B 15/246; A24B 15/26; A24B 15/303; A61K 36/3482

(57) ABSTRACT

Provided is a composition comprising multiple cannabis storage cavities and/or multiple cannabis secretory cells, wherein at least 20% of said storage cavities and/or at least 20% of said secretory cells are intact and wherein each of said intact storage cavities and/or each of said intact secretory cells contains therein at least one cannabinoid. Further provided are products comprising the composition and methods of use thereof.

### CROSS-REFERENCE TO RELATED APPLICATIONS

The present application gains priority from U.S. Provisional Patent Application Ser. No. 63/320,222 filed Mar. 16, 2022 and 63/321,782 filed Mar. 21, 2022, both of which are incorporated by reference as if fully set-forth herein.

### FIELD OF THE INVENTION

The present invention relates to the field of cannabinoid-containing compositions, and more specifically to such compositions comprising multiple cannabis storage cavities and/or multiple cannabis secretory cells containing cannabinoids.

### SUMMARY OF THE INVENTION

According to an aspect of some embodiments of the present invention, there is provided a composition comprising multiple cannabis storage cavities and/or multiple cannabis secretory cells, wherein at least 20% of said storage cavities and/or at least 20% of said secretory cells are intact and wherein each of said intact storage cavities and/or each of said intact secretory cells contains therein at least one cannabinoid.

According to an aspect of some embodiments of the present invention, there is provided product comprising at least one composition as disclosed herein.

According to an aspect of some embodiments of the present invention, there is provided a vaporizer composition comprising the composition as disclosed herein.

According to an aspect of some embodiments of the present invention, there is provided a method for providing at least one cannabinoid to a subject in need thereof, the method comprising administering to said subject a composition comprising multiple cannabis storage cavities and/or multiple secretory cells, wherein at least 20% of said storage cavities and/or at least 20% of said secretory cells are intact and wherein each of said intact storage cavities and/or said intact secretory cells contains therein at least one cannabinoid.

According to an aspect of some embodiments of the present invention, there is provided a method for providing at least one cannabinoid to a subject, comprising extracting the composition as disclosed herein with a solvent to form a solvent-comprising extract, optionally removing solvent from said extract to provide a separated extract, and administering said solvent-comprising extract or said separated extract to said subject.

### DETAILED DESCRIPTION OF THE INVENTION

The present invention relates to a composition comprising multiple cannabis storage cavities containing cannabinoids.

Unless otherwise defined, all technical and scientific terms used herein have the same meaning as commonly understood by one of ordinary skill in the art to which this invention belongs.

The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

As used herein, the term “treating” includes ameliorating, mitigating, and reducing the instances of a disease or condition, or the symptoms of a disease or condition.

As used herein, the term “therapeutically effective amount” means the amount of an active substance that, when administered to a subject for treating a disease, disorder, or other undesirable medical condition, is sufficient to have a beneficial effect with respect to that disease, disorder, or condition. The therapeutically effective amount will vary depending on the chemical identity and formulation form of the active substance, the disease or condition and its severity, and the age, weight, and other relevant characteristics of the patient to be treated. Determining the therapeutically effective amount of a given active substance is within the ordinary skill of the art and typically requires no more than routine experimentation.

As used herein, the term “cannabis storage cavity” refers to an extracellular cannabis storage cavity produced by cultured cannabis plant cells.

As used herein, the term “secretory cell” means cannabis secretory cells according to Livingston, et. al. The Plant Journal, 2020, 101, 37-56.

Unless otherwise indicated, all numbers expressing quantities, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”. As used herein, the terms “water content” and “moisture content” are used interchangeably.

Unless indicated otherwise, percent is weight percent and ratio is weight/weight ratio.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a composition comprising multiple cannabis storage cavities and/or multiple cannabis secretory cells, wherein at least 20% (such as at least 20%, at least 30%, at least 40%, at least 50%, at least 60%, at least 70%, at least 80%, or at least 90%) of the storage cavities and/or at least 20% of the secretory cells are intact or substantially intact, and wherein each of the intact storage cavities and/or each of the intact secretory cells contains therein at least one cannabinoid. According to some embodiments, cells that are not intact or substantially intact contain at least one cannabinoid and/or at least one terpene. According to some embodiments, cells that are not intact or substantially intact contain at least one cannabinoid and at least one terpene.

As used herein, the term “intact” means essentially unbroken and complete.

According to some embodiments, at least 50% of the total number of cavities (such as 50%, about 55%, about 60%, about 65%, about 70%, about 75%, about 80%, about 85%, about 90%, about 95% or even about 100%) of the cavities are of a size in the range of about 1-100 μm, such as about 1, about 10, about 20, about 30, about 40, about 50, about 60, about 70, about 80, about 90 or about 100 μm.

According to some embodiments, the storage cavities and/or secretory cells form at least about 30% wt of the composition, such as about 30% wt, about 35% wt, about 40% wt, about 45% wt, about 50% wt, about 55% wt, about 60% wt, about 65% wt, about 70% wt, about 75% wt, about 80% wt, about 85% wt, about 90% wt, about 95% wt or about 100% wt.

According to some embodiments, at least about 50% of the cannabinoid is contained within said storage cavities and/or said secretory cells, such as about 50%, about 55%, about 60%, about 65%, about 70%, about 75%, about 80%, about 85%, about 90%, about 95% or about 100%.

According to some embodiments, at least 50% wt (such as at least 50% wt, at least 60% wt, at least 70% wt, at least 80% wt, or at least 90% wt) of a total cannabinoid content of the composition is in decarboxylated form.

According to some embodiments, the at least one cannabinoid in each of the multiple storage cavities and/or in each of the secretory cells is selected from the group consisting of THC, THCa, CBD, CBDa, CBG, CBGa, CBC, CBCa, CBN, CBNa, THCV, THCVa and combinations thereof. According to some embodiments, the same cannabinoid is present in each of the multiple storage cavities.

According to some embodiments, each of the storage cavities and/or each of the secretory cells further contains therein at least one terpene, such as one, two, three, four, five or more than five terpenes. According to some embodiments, the at least one terpene comprises at least one monoterpene and at least one sesquiterpene, wherein a total monoterpenes to total sesquiterpenes weight/weight ratio in the composition is greater than 1 (such as greater than 1, greater than 2, greater than 3, greater than 4 or greater than 5). According to some embodiments, the at least one terpene comprises at least one monoterpene and at least one sesquiterpene, wherein the total sesquiterpenes to total monoterpenes weight/weight ratio is greater than 1 (such as greater than 1, greater than 2, greater than 3, greater than 4 or greater than 5).

According to some embodiments, the composition comprises discrete particles, wherein the storage cavities and/or the secretory cells are present within the discrete particles.

According to some embodiments, at least 20% of the discrete particles have a size in the range between about 0.5 mm and about 4 mm, such as about 0.5 mm, about 1.0 mm, about 1.5 mm, about 2.0 mm, about 2.5 mm, about 3.0 mm, about 3.5 mm or about 4.0 mm. According to some such embodiments, the discrete particles are formed by a method comprising granulating the storage cavities and/or the secretory cells.

According to some embodiments, the total cannabinoid concentration of the composition at least 40% (such as at least 40%, at least 50%, at least 60%, at least 70%, at least 80% or at least 90%) of the total weight of the composition.

According to some embodiments, the total cannabinoid concentration of the storage cavities and/or of the cannabis secretory cells is at least 40% (such as at least 40%, at least 50%, at least 60%, at least 70%, at least 80% or at least 90%) of the total weight of the composition.

According to an aspect of some embodiments of the present invention, there is provided a product comprising the composition as disclosed herein.

According to some embodiments, the product further comprises a carrier. According to some embodiments, the carrier is selected from the group consisting of edible oils, pharmaceutical oils, pharmaceutical excipients, cannabis plant material, tobacco and combinations thereof. According to some embodiments, the carrier comprises ground cannabis, ground tobacco or combinations thereof and the product is in the form of a cigarette.

According to some embodiments, the product further comprises a cannabis extract which is not contained within the storage cavities and/or within the secretory cells. According to some such embodiments, the cannabis extract is produced by extracting cannabis with a solvent selected from the group consisting of supercritical CO2, ethanol, isopropanol, hydrocarbons, vegetable oils and a mixture thereof.

According to some embodiments, the product further comprises at least one isolated cannabinoid which is not contained within the storage cavities and/or within the secretory cells. As used herein, the term “isolated cannabinoid” means cannabinoid having a purity of at least 90% wt, such as 90% wt, about 95% wt, about 98% wt or about 100% wt.

According to some embodiments, the product further comprises at least one terpene which is not contained within the storage cavities and/or within the secretory cells.

According to some embodiments, the at least one composition comprises at least two compositions, such as two, three, four, five, six, or more than six compositions, wherein a first composition comprises a first cannabinoid and/or terpene content and a second composition comprises a second cannabinoid and/or terpene content, wherein the first composition is different from the second composition. According to some embodiments, the cannabinoids of the first composition are different from those of the second composition. According to some embodiments, the cannabinoids of the first composition are the same as those of the second composition but are present at different concentrations.

According to some embodiments, the at least one composition comprises at least two compositions, wherein a first composition has a first degree of decarboxylation and a second composition has a second degree of decarboxylation, wherein the first degree of decarboxylation is different from the second degree of decarboxylation.

According to some embodiments, the at least one composition comprises at least two compositions, such as two, three, four, five, six or more than six compositions, wherein a first composition comprises a first content of intact cavities and/or intact cells and a second composition comprises a second content of intact cavities and/or intact cells, wherein the first content of intact cavities and/or intact cells is different from the second content of intact cavities and/or intact cells. According to some such embodiments, the concentration of intact cavities and/or intact cells in the first composition is different from the concentration of intact cavities and/or intact cells in the second composition. According to some such embodiments, the concentration of intact cavities and/or intact cells in the second composition is higher than that concentration of intact cavities and/or intact cells in the first composition, such that the first composition provides a faster release rate of cannabinoids and/or terpenes, and the second composition provides a slower and more sustained release of cannabinoids and/or terpenes.

According to some embodiments, the product is provided in the form of a gel capsule.

According to some embodiments, the product is selected from the group consisting of foods, beverages, shakes, medications and nutraceuticals. According to some such embodiments, the product is a food, a beverage and/or a shake comprising at least 50% wt of water.

As used herein, the term “nutraceutical” refers to a substance that is a food or food ingredient and provides medical or health benefits, including the prevention and treatment of a disease. According to some embodiments, the product is provided in a form selected from the group consisting of patches, tablets, disintegrating tablets, coated tablets, capsules, creams, powders and film-forming compositions. According to some such embodiments, the product is configured for delivery by a route selected from the group consisting of transdermal, transmucosal, sublingual, oral, suppository, vaginal and combinations thereof.

According to an aspect of some embodiments of the present invention, there is provided a vaporizer composition comprising the composition as disclosed herein.

According to an aspect of some embodiments of the present invention, there is provided a method for providing a composition as disclosed herein wherein at least 50% wt the total cannabinoid content of the composition is in decarboxylated form, the method comprising providing a composition comprising multiple cannabis storage cavities and/or multiple cannabis secretory cells, wherein at least 20% of the storage cavities and/or at least 20% of the secretory cells are intact and wherein each of the intact storage cavities and/or each of the intact secretory cells contains therein at least one cannabinoid, and decarboxylating at least 50% of the at least one cannabinoid at a temperature and for a duration that reduces the content of the intact cells by less than 50%, less than 40%, less than 30%, less than 20% or less than 10%. According to some such embodiments, the decarboxylating is conducted at a temperature of less than 80° C., less than 70° C., less than 60° C. or even less than 50° C.

According to an aspect of some embodiments of the present invention, there is provided a method for providing at least one cannabinoid to a subject in need thereof, the method comprising administering to the subject a composition comprising multiple cannabis storage cavities and/or multiple secretory cells, wherein at least 20% of the storage cavities and/or at least 20% of the secretory cells are intact and wherein each of the intact storage cavities and/or the intact secretory cells contains therein at least one cannabinoid.

According to some embodiments, the cannabinoid is liberated over a period of at least 4 hours, such as 4, about 5, about 6, about 7, about 8 or even more than about 8 hours.

According to an aspect of some embodiments of the present invention, there is provided a method for providing at least one cannabinoid to a subject, comprising extracting the composition as disclosed herein with a solvent to form a solvent-comprising extract, optionally removing solvent from the extract to provide a separated extract, and administering the solvent-comprising extract or the separated extract to the subject.

According to some embodiments, the method further comprises grinding, pulverizing and/or freezing the composition prior to the extracting. As used herein, the term “freezing” means maintaining the composition at a temperature of less than 0° C. for at least one hour.

According to some embodiments, the method further comprises decarboxylating at least 50% of the at least one cannabinoid prior to said extracting. According to some such embodiments, the decarboxylating is conducted at a temperature of at least 80° C., at least 90° C., at least 100° C. or at least 110° C.

### EXAMPLES

### Example 1: Production of a Composition Comprising Multiple Cannabis Storage Cavities and Multiple Cannabis Secretory Cells

A cannabis plant material was obtained from a licensed grower in Israel. Analysis showed that it contained about 9% wt of THC. About 1 kg of that material was introduced into a trichomes separating machine (a rotating box equipped with a net having pore size of 180 μm). The machine was operated for an hour. The product obtained after each 10 minute period was collected separately. Each product was analyzed. The first product contained 44.9% wt of total THC. Total THC was calculated by combining the THC content with 0.88 times that THCA content. As the process continued, the total THC concentration declined due to increase in the fraction of trichomes-poor particles. Observation under a microscope confirmed that the majority of the trichomes in the products were intact.

### Example 2: Production of Granules

5 grs of a product, produced according to Example 1 and containing about 42% THC, were wetted with 1 ml of 95% ethanol and granulated to form granules of about 1 to 2 millimeter size. The ethanol content of the granules was removed by heating in an oven at 60° C.

### Example 3: Production of THC-Enriched Cigarette

Cannabis inflorescence comprising 11% moisture was kept in a freezer for 48 hours and then ground to form ground inflorescence with particle size of about 2 mm. 9 gr of the ground inflorescence were blended with 1 gr of granules formed according to Example 2. A uniform blend was formed. A sample of the THC-enriched blend was analyzed and found to contain about 14% wt total THC.

A cigarette was produced by rolling 0.5 gr of the THC-enriched blend in a rolling paper. The cigarette was smoked, showing even burning.

### Example 4: Extraction With Ethanol

10 grs of a product, produced according to Example 1 and containing about 39% THC were mixed in a test tube with 100 gr of 95% ethanol at ambient temperature for 30 minutes. The tube was then centrifuged and the clear ethanol extract was separated. The extract was treated at 60° C. in a rotavapor under vacuum for complete removal of the ethanol. A yellow ethanol-free extract was formed. Analysis showed that the total THC concentration was about 80% wt. The majority of it (>90%) was in the form of THCA.

### Example 5: Decarboxylation of an Extract Formed by Extraction With Ethanol

6 gr of an extract formed according to Example 4 were kept at 100° C. for 25 minutes. Analysis of the extract after cooling showed that >90% of the total THC was converted into the decarboxylated form—THC.

### Example 6: Extraction With Olive Oil

3.3 grs of a product, produced according to Example 1 and containing about 44% THC were mixed in a test tube with 6.6 grs of therapeutic-grade olive oil. The test tube was heated to 70 C.° and mixing was continued for 30 min. The tube was then centrifuged and the clear olive-oil extract was separated. Analysis showed that the total THC concentration was about 17% wt.

### Example 7: Decarboxylation of an Extract Formed by Extraction With Olive Oil

3 gr of an extract formed according to Example 6 were kept at 90° C. for 3 hours. Analysis of the extract after cooling showed that 85% of the total THC was converted into the decarboxylated form—THC.

1. A composition comprising multiple cannabis storage cavities and/or multiple cannabis secretory cells, wherein at least 20% of said storage cavities and/or at least 20% of said secretory cells are intact, wherein each of said intact storage cavities and/or each of said intact secretory cells contains therein at least one cannabinoid and wherein at least 50% of the cannabinoid is contained within said storage cavities and/or said secretory cells.

2. The composition of claim 1, wherein said storage cavities and/or said secretory cells form at least 30% wt of the composition.

3. (canceled)

4. The composition of claim 1, wherein at least 50% wt of a total cannabinoid content of said composition is in decarboxylated form.

5. The composition of claim 1, wherein said at least one cannabinoid in each of said multiple storage cavities and/or in each of said secretory cells is selected from the group consisting of THC, THCa, CBD, CBDa, CBG, CBGa, CBC, CBCa, CBN, CBNa, THCV, THCVa and combinations thereof.

6. (canceled)

7. The composition of claim 1, wherein each of said storage cavities and/or each of said secretory cells further comprises at least one monoterpene and at least one sesquiterpene and wherein a total monoterpenes to total sesquiterpenes weight/weight ratio in said composition is greater than 1.

8. The composition of claim 1, wherein each of said storage cavities and/or each of said secretory cells further comprises at least one monoterpene and at least one sesquiterpene and wherein the total sesquiterpenes to total monoterpenes weight/weight ratio is greater than 1.

9. The composition of claim 1, comprising discrete particles, wherein said storage cavities and/or said secretory cells are present within said discrete particles.

10. The composition of claim 9, wherein at least 20% of said discrete particles have a size in the range between 0.5 mm and 4 mm.

11-12. (canceled)

13. A product comprising at least one composition according to claim 1.

14. (canceled)

15. The product of claim 13, further comprising a carrier, wherein said carrier is selected from the group consisting of edible oils, pharmaceutical oils, pharmaceutical excipients, cannabis plant material, ground cannabis, tobacco, ground tobacco and combinations thereof.

16. The product of claim 15, wherein said carrier comprises ground cannabis, ground tobacco or combinations thereof and said product is in the form of a cigarette.

17. The product of claim 13, further comprising a compound which is not contained within said storage cavities and/or within said secretory cells, wherein said compound is selected from the group consisting of cannabis extract, at least one isolated cannabinoid, at least one terpene and combinations thereof.

18-19. (canceled)

20. The product of claim 13, wherein said at least one composition comprises at least two compositions,
(a) wherein a first composition comprises a first cannabinoid and/or terpene content and a second composition comprises a second cannabinoid and/or terpene content,
(b) wherein a first composition has a first degree of decarboxylation and a second composition has a second degree of decarboxylation, wherein said first degree of decarboxylation is different from said second degree of decarboxylation, or
(c) wherein a first composition comprises a first content of intact cavities and/or intact cells and a second composition comprises a second content of intact cavities and/or intact cells, wherein said first content of intact cavities and/or intact cells is different from said second content of intact cavities and/or intact cells.

21-23. (canceled)

24. The product of claim 13, wherein said product is selected from the group consisting of foods, beverages, shakes, medications and nutraceuticals.

25. (canceled)

26. The product of claim 13, in a form selected from the group consisting of patches, tablets, disintegrating tablets, coated tablets, capsules, gel capsules, cigarette, creams, powders and film-forming compositions.

27. The product of claim 26, configured for delivery by a route selected from the group consisting of transdermal, transmucosal, sublingual, oral, suppository, vaginal and combinations thereof.

28. A vaporizer composition comprising the composition of claim 1.

29. A method for providing the composition of claim 4, comprising providing a composition comprising multiple cannabis storage cavities and/or multiple cannabis secretory cells, wherein at least 20% of said storage cavities and/or at least 20% of said secretory cells are intact and wherein each of said intact storage cavities and/or each of said intact secretory cells contains therein at least one cannabinoid, and decarboxylating at least 50% of said at least one cannabinoid at a temperature and for a duration that reduces the content of the intact cells by less than 50%.

30. The method of claim 29, wherein said decarboxylating is conducted at a temperature of less than 80° C.

31. A method for providing at least one cannabinoid to a subject in need thereof, the method comprising administering to said subject a composition comprising multiple cannabis storage cavities and/or multiple secretory cells, wherein at least 20% of said storage cavities and/or at least 20% of said secretory cells are intact and wherein each of said intact storage cavities and/or said intact secretory cells contains therein at least one cannabinoid.

32-36. (canceled)
